(19) United States
(12) Patent Application Publication
(10) Pub. No.: US 2023/0241135 A1
(43) Pub. Date: Aug. 3, 2023

(54) A COMPOSITION FOR REDUCING THE RISK OF URINARY TRACT INFECTION AND VAGINAL INFECTION IN WOMEN

(71) Applicants: Kira HALAK, Columbia, MD (US); DSM IP Assets B.V., Heerlen (NL)

(72) Inventors: Kira HALAK, Columbia, MD (US); Seema K. MODY, Columbia, MD (US)

(21) Appl. No.: 18/010,130

(22) PCT Filed: Jun. 15, 2021
(86) PCT No.: PCT/US2021/037482

§ 371 Date: Dec. 13, 2022

Related U.S. Application Data
(60) Provisional application No. 63/039,809, filed on Jun. 16, 2020.

(51) Int. Cl. A61K 35/747; A61K 35/745; A61K 35/76; A61P 13/02; A61P 31/04; A61K 9/00

(52) U.S. Cl. CPC A61K 35/747; A61K 35/745; A61K 35/76; A61P 13/02; A61P 31/04; A61K 9/0053; A61K 2035/115

(57) ABSTRACT

The present invention is directed to a non-probiotic composition for treating both urinary tract infection and vaginal infection, and to restore female microbiota. The above composition comprises at least one probiotic strain and at least one phage strain. The present invention is also directed to a method for using the above composition.

### BACKGROUND OF THE INVENTION

Due to widespread antibiotic use and a disrupted female microbiome, many women have recurring infections or cycle between recurring urinary tract infections (UTI) and vaginal infections.

Both urinary tract and vaginal infections are common concerns for women. Urinary tract infections are among the most prevalent bacterial infections in the US. Half of women have at least one UTI by age 32 with recurrence rates up to 46% within 1 year (Dielubanza and Schaeffer. Med. Clin. North Am. 2011; 95(1):27-41, Ferri FF. Ferri's Clinical Advisor 2019: Philadelphia, Pa: Elsevier.; 2019).

Escherichia coli is the bacterial pathogen in 85% of UTI cases (Ferri FF. Ferri's Clinical Advisor, 2019). Ascending infection via the urethra with bacterial flora from the genital and gastrointestinal tracts is the major pathway for UTI in women (Ferri FF. Ferri's Clinical Advisor, 2019). It is generally accepted that uropathogenic E. coli (UPEC) migrate from the gastrointestinal tract to the periurethral area and up the urethra. Gastrointestinal E. coli abundance is also a risk factor for urinary tract infections (Magruder et al. Nature Communications. 2019;10(1):5521).

Recurrent UTIs represent repeated movement of UPEC strains from the gut to the urinary tract (Chen, Wu et al. Sci Transl Med. 2013;5(184):184ra60).

Approximately 30% of women in the US have vaginal bacterial vaginosis (BV). (Koumans, Sternberg et al. Sex Transm. Dis. 2007; 34(11):864-9). On average, 58% of women with BV who are treated with a seven-day course of antibiotics will have a recurrence within a year (Bradshaw, Morton et al. The Journal of Infectious Diseases. 2006; 193(11):1478-86).

Approximately 70-75% of women will experience a “yeast infection”, or episode of vulvovaginal candidiasis (VVC) in their lifetimes. 50% of initially infected women will suffer a second VVC event and 5-10% of all women will develop recurrent VVC (Goncalves, Ferreira et al. Critical reviews in microbiology. 2016; 42(6):905-27).

Yeast infections are caused by an overgrowth of Candida species, primarily of Candida albicans. Bacterial vaginosis (BV) is a clinical condition caused by an overgrowth of vaginal bacteria, such as Gardnerella.

A healthy vaginal microbiota is dominated by lactic acid and hydrogen peroxide producing lactobacilli. These microorganisms are essential to prevent colonization and overgrowth of pathogenic organisms such as Candida or Gardnerella (Borges et al. Archives of gynecology and obstetrics. 2014; 289(3):479-89).

The presence of high numbers of lactic acid bacteria is typically equated with health while an absence or low count (vaginal dysbiosis) is an abnormal state (Hickley et al. Transl Res. 2012; 160(4):267-82).

Due to the movement of bacteria, the “female microbiota” encompasses more than only the vaginal microbiota and instead more broadly includes the gastrointestinal, urinary tract, perineum and vaginal microbiotas. The health of the “female microbiota” is critical for prevention of infection in the vagina and urinary tract.

The equilibrium of the female microbiota routinely experiences various chronic and acute challenges caused by human behaviors, such as birth control, sexual intercourse, vaginal lubricants and douching. In addition, antibiotic usage (for BV or UTI or other infections) disturbs the protective female microbiota which increases the chances of recurring BV, UTI or an overgrowth of Candida.

Not only do antibiotics disturb the feminine microbiota, they also contribute to the development of antibiotic resistant bacteria. Since UTIs account for approximately 15% of all community-prescribed antibiotics in the United States, they have a key role for direct antibiotic selection pressure. (Leitner, Sybesma et al. BMC Urol. 2017;17(1):90.) Due to the overuse of antibiotics, organisms once sensitive to a number of antimicrobial agents are now increasingly resistant, making effective management of UTI and pyelonephritis more challenging and potentially more dangerous (Ferri FF. Ferri's Clinical Advisor 2019). Multidrug-resistant uropathogenic Escherichia coli (UPEC) is increasing on a worldwide scale.(Nishikawa, Yasuda et al. Archives of virology. 2008; 153(3):507-15.) Prevention and alternatives to antibiotics are needed.

What is needed, but presumably lacking, is a method of preventing or treating recurring urinary tract infection and vaginal infection in women that is alternative to antibiotics use.

### SUMMARY OF THE INVENTION

The present invention is directed to a composition comprising at least one probiotic which has anti-bacterial vaginosis and/or anti-candida activity and at least one phage which has anti-pathogenic E. coli activity.

In one embodiment, the probiotic comprises a prokaryote, eukaryote, or archaebacteria probiotic. In another embodiment, the probiotic comprises at least one of any suitable strain or subspecies of Enterococcus, Streptococcus, Lactobacillus, Lactococcus, Bifidobacterium, or Saccharomyces. In another embodiment, the probiotic comprises Lactobacillus crispatus strain LBV 88, Lactobacillus rhamnosus strain LBV 96, Lactobacillus gasseri strain LBV 150N, Lactobacillusjensenii strain LBV 116.

In one embodiment, the phage comprises at least one of suitable strain in the families of Myoviridae and Siphoviridae. In another embodiment, the phage comprises Myoviridae strains LHO1, T4D, and LL12, and Siphoviridae strain LL5.

In one embodiment, the composition is formulated in a dosage which is sufficient for improving or maintaining the urinary tract health and vaginal health of a woman.

In another embodiment, the composition is formulated in a dosage which is sufficient for treating, reducing the duration and severity, or reducing the risk of urinary tract infection and vaginal infection of a woman.

In another embodiment, the composition is formulated in a dosage which is sufficient for improving the vaginal and/or fecal microbiota of a woman.

In another embodiment, the composition is formulated in a dosage which is sufficient for increasing the population of vaginal lactobacilli of a woman.

In one embodiment, the probiotic in said composition is formulated in a dosage of at least 5 billion CFUs per day.

In one embodiment, the phage in said composition is formulated in a dosage of at least 5×10⁵ PFU per day.

In one embodiment, the composition is formulated for oral administration.

In one embodiment, the composition is formulated as a dietary supplement, a food, a medical food, or a pharmaceutical.

The present invention is also directed to a kit suitable for administering a composition orally to a human, comprising in a packet any one of the above described composition and wherein the composition is in the form of powder, and instructions for how to use the kit.

The present invention is also directed to a method for treating woman's urinary tract infection and/or vaginal infection of a woman, comprising orally administering to the woman any one of the above described composition.

The present invention is also directed to a method for reducing the duration of severity of woman's urinary tract infection and/or vaginal infection of a woman, comprising orally administering to the woman any one of the above described composition.

The present invention is also directed to a method for improving the vaginal and/or fecal microbiota of a woman, comprising orally administering to the woman the composition of any one of the above described composition.

The present invention is also directed to a method for increasing the population of vaginal lactobacilli of a woman, comprising orally administering to the woman the composition of any one of the above described composition.

The present invention is also directed to a method for improving or maintaining the urinary tract health and vaginal health of a woman, comprising orally administering to the woman the composition of any one of the above described composition.

### BRIEF SUMMARY OF DRAWINGS

FIG. 1 is a graph showing the impact of phage blend on the growth of probiotics when the probiotic blend is co-incubated with E. coli and the phage blend.

### DETAILED DESCRIPTION OF THE INVENTION

In the present invention, the inventors have made a combination of compounds comprising at least one probiotic which has anti-vaginal infection activity and at least one phage which has anti-pathogenic E. coli activity. This combination of compounds has a spectacular and unexpected selectivity towards inhibiting uropathogenic E. coli strains which cause urinary tract infection as well as inhibiting pathogenic vaginal microbes, such as Candida and Gardnerella, which cause vaginal bacterial vaginosis and yeast infection. The combination of compounds disclosed in the application have a considerably less inhibitory effect towards non-pathogenic E. coli strains or other beneficial commensal micro-organisms that are present in a healthy female microbiota. The probiotic strains described herein are those which have shown the beneficial effect of inhibiting bacterial vaginosis and vaginal yeast infections competing with those harmful microorganisms in the vaginal tract. The at least one phage described herein are those which has shown the benefit of inhibiting uropathogenic E. coli strains but does not inhibit the growth of nonpathogenic E. coli strains. In one embodiment, the above combination of compounds comprise more than one probiotic strain and more than one phage, for example, a mixture of probiotic strains and a mixture of phages.

It was surprisingly found by the inventors that the above combination of probiotic mixture and phage mixture possess a synergistic effect in which the combined effect on inhibiting urinary tract infection and vaginal infection is more significant than the use of either the probiotic mixture or phage mixture alone. Particularly, it was found that the above combination of probiotic mixture and phage mixture has an inhibitory effect on pathogenic E. coli that is much higher than the same dosage of phage mixture alone. It was also found that the above combination of probiotic mixture and phage mixture has an inhibitory effect on pathogenic vaginal microbes that is much higher than the same dosage of probiotic mixture alone. It was believed that such synergistic effect was caused by the use of both phage mixture and probiotic mixture in the combination instead of alone.

The discovery presents itself towards new uses for modulating female microbiomes. By preventing or treating conditions or diseases associated with female microbiome, the combination of probiotic mixture and phage mixture disclosed in this application demonstrate its effectiveness in rebalancing the female microbiome. The use of the above composition improves gastrointestinal, urinary tract, perineum and vaginal microbiotas. In fact, the composition not only prevents infection in the gastrointestinal, urinary tract and vagina, it also improves the overall health of female microbiota. A healthy female microbiota has a significantly lower chance of developing female urinary tract infection and vaginal infection.

As a result, the invention provides an alternative over previous methods of treatment, such as broad spectrum antibiotics. The invention presents a significant advantage over treatment by antibiotics, which do not have a high degree of specificity. For example, antibiotic usage for treating urinary tract infection and vaginal infection is known to kill not only detrimental bacteria but also beneficial bacteria, and thus disturbs the protective female microbiota. Antibiotic usage reduces the ability of the female microbiota to suppress the overgrowth of uropathogenic E. coli, bacterial vaginosis and Candida, and thus leads to recurring urinary tract infection and vaginal infection.

Replacing antibiotic usage or at least reducing the amount of antibiotic usage with the composition disclosed in this invention will help to treat urinary tract infection and vaginal infection and avoid the disadvantage of antibiotic use such as the disruption of the beneficial microbiome. Furthermore, it helps to prevent the formation of antibiotic resistant bacteria. It helps to maintain the equilibrium of the female microbiota and thus deter the recurrence of urinary tract infection and vaginal infection.

Thus, in one embodiment, the present invention is directed to a composition which comprises at least one probiotic which has anti-vaginal infection activity and at least one phage which has anti-pathogenic E. coli activity (anti-UTI). In one embodiment, this composition helps to treat urinary tract infection and vaginal infection. In another embodiment, the composition helps to deter the recurrence of urinary tract infection and vaginal infection. In another embodiment, the composition helps to restore female microbiota.

In one embodiment, the above composition of combined probiotic mixture and phage mixture has an anti-pathogenic E. coli activity that is at least 1, at least 2, at least 3, at least 4, at least 5, or at least 6 log unit (multiple of 10) higher than a control composition which contains the same amount of either the probiotic mixture or the phage mixture alone.

In another embodiment, the above composition of combined probiotic mixture and phage mixture has an anti-vaginal infection activity that is at least 1, or at least 2 fold (multiple of 2) higher than a control composition which contains the same amount of either the probiotic mixture alone.

According to one aspect of the present invention, the above probiotic mixture can provide the beneficial effect of preventing and treating vaginal bacterial vaginosis and yeast infection, and restoring the normal microflora balance in women's vaginal tract.

According to another aspect of the present invention, the above phage mixture can specifically lyse uropathogenic E. coli strains but do not inhibit the growth of other non-pathogenic E. coli strains or bacteria strains.

According to one aspect of the present invention, the probiotic used in the composition of the present invention comprise a mixture of one or more different probiotic strains. In one embodiment, the mixture of different probiotic strains comprises prokaryote, eukaryote, or archaebacteria probiotic strains. In another embodiment, the mixture of different probiotic strains comprises at least one of any suitable strain or subspecies of Enterococcus, Streptococcus, Lactobacillus, Lactococcus, Bifidobacterium, or Saccharomyces.

In one embodiment, the mixture of different probiotic strains are those strains which are known to have a beneficial effect in the prevention and treatment of vaginal infection. In another embodiment, the mixture of different probiotic strains are those strains which are able to enhance intestinal function, stimulate the immune system, reduce inflammation, and diminish the population of harmful microorganisms in the vaginal tract. In another embodiment, the mixture of different probiotic strains are those strains which inhibit the growth of pathogenic vaginal yeast, such as Candida, and the growth of vaginal bacteria, such as Gardnerella.

In a specific embodiment, the mixture of different probiotic strains comprises Lactobacillus crispatus strain LBV 88, Lactobacillus rhamnosus strain LBV 96, Lactobacillus gasseri strain LBV 150N, and Lactobacillusjensenii strain LBV 116.

In one embodiment, the dosage of the mixture of different probiotic strains in the composition of this invention is formulated in an amount that is to reduce the population of harmful microorganisms in the vaginal tract.

Probiotic concentration in the contemplated probiotic mixture can range from 10 million cfu/gram to 100 billion cfu/gram, from 10 million to 50 million cfu/gram, more preferably from 50 million to 100 million cfu/gram, from 100 million to 500 million cfu/gram, from 500 million to 1 billion cfu/gram, from 1 billion to 5 billion cfu/gram, from 5 billion to 10 billion cfu/gram, from 10 billion to 15 billion cfu/gram, from 15 billion to 20 billion cfu/gram, from 20 billion to 25 billion cfu/gram, from 25 billion to 30 billion cfu/gram, from 30 billion to 35 billion cfu/gram, from 35 billion to 40 billion cfu/gram, from 40 billion to 45 billion cfu/gram, from 45 billion to 50 billion cfu/gram, from 50 billion to 55 billion cfu/gram, from 55 billion to 60 billion cfu/gram, from 60 billion to 65 billion cfu/gram, from 65 billion to 70 billion cfu/gram, from 70 billion to 75 billion cfu/gram, from 75 billion to 80 billion cfu/gram, from 80 billion to 85 billion cfu/gram, from 85 billion to 90 billion cfu/gram, from 90 billion to 95 billion cfu/gram, from 95 billion to 100 billion cfu/gram.

According to another aspect of the present invention, the phage component of the composition comprises a mixture of one or more different phage strains which can specifically lyse uropathogenic E. coli but do not inhibit the growth of non-pathogenic E. coli or other residential bacteria in the urinary tract, or more broadly, in the female microbiota.

In one embodiment, the mixture of phage strains comprises Myoviridae strains LHO1, T4D, and LL12, and Siphoviridae strain LL5.

The concentration of the contemplated phage mixture ranges from 1×10⁴ pfu/gram to 2×10⁸ pfu/gram, from 5×10⁴ pfu/gram to 2×10⁸ pfu/gram, 1×10⁵ pfu/gram to 2×10⁸ pfu/gram, 5×10⁵ pfu/gram to 2×10⁸ pfu/gram, from 1×10⁶ pfu/gram to 2×10⁸ pfu/gram, from 5×10⁶ pfu/gram to 2×10⁸ pfu/gram, from 1×10⁷ pfu/gram to 2×10⁸ pfu/gram, from 5×10⁷ pfu/gram to 1×10⁸ pfu/gram.

In another embodiment, the above composition is formulated for oral administration. In another embodiment, the composition is formulated as a dietary supplement, a food, or a pharmaceutical.

It is known in the art that compositions for treating vaginal infections and urinary tract infections can be administered directly into vaginal tract or urinary tract. However, the inventor of the present invention has developed a convenient way of administration a composition targeting at infections in woman's vaginal tract and urinary tract via oral ingestion. It avoids the inconvenience of privacy requirement of the existing method and makes it possible for the woman to take the composition anywhere at any time. The composition developed by the inventor of the present invention can pass through gastrointestinal tract and reach both vaginal tract and urinary tract with sufficient count of probiotic strains and phage strains.

In one embodiment, the composition is enteric coated to protect it from the damage caused by the gastrointestinal tract. In another embodiment, the probiotic strains in the composition are strains which can withstand the acid environment in the gastrointestinal tract and vaginal tract. Such design allows sufficient number of probiotic strains and phage strains to reach both vaginal tract and urinary tract with minimal degradation caused by the gastrointestinal tract.

The present invention is also directed to a kit suitable for administering a composition orally to a human, comprising in a packet the above composition which comprises at least one probiotic and at least one phage which has anti-pathogenic E. coli activity, and instructions for how to use the composition. In one embodiment, the kit is for treating urinary tract infection and vaginal infection. Making the composition in the form of a kit makes it easier for distribution and use of the composition.

In some embodiments, the dose amount in the kit is more than 100 mg per capsule, more than 150 mg per capsule, more than 200 mg per capsule, more than 250 mg per capsule, more than 350 mg per capsule, more than 400 mg per capsule, more than 450 mg per capsule, more than 500 mg per capsule, or more than 550 mg per capsule, or more than 600 mg per capsule. In some other embodiments, the dose amount in the kit is between 100 mg and 700 mg per capsule, between 200 mg and 600 mg per capsule, between 300 mg and 600 mg per capsule, between 400 mg and 600 mg per capsule, between 300 mg and 500 mg per capsule, 400 mg and 500 mg per capsule, or between 350 mg and 500 mg per capsule.

In some embodiments, the amount of probiotic mixture in the kit is no less than 1 billion CFU, no less than 2 billion CFU, no less than 3 billion CFU, no less than 4 billion CFU, no less than 5 billion CFU, or no less than 6 billion CFU. In some embodiments, the amount of probiotic mixture in the kit is between 1 billion CFU and 6 billion CFU, between 2 billion CFU and 5 billion CFU, between 3 billion CFU and 6 billion CFU, between 4 billion CFU and 6 billion CFU, or between 4.5 billion CFU and 5.5 billion CFU.

In some embodiments, the amount of phage mixture in the kit is no less than 1×10⁵ PFU, no less than 2×10⁵ PFU, no less than 3×10⁵ PFU, no less than 4×10⁵ PFU, no less than 5×10⁵ PFU, no less than 6×10⁵ PFU. In some embodiments, the amount of phage mixture in the kit is between 1×10⁵ and 6×10⁵ PFU, between 2×10⁵ and 6×10⁵ PFU, between 3×10⁵ and 6×10⁵ PFU, between 4×10⁵ and 6×10⁵ PFU, or between 4×10⁵ and 5×10⁵ PFU.

In yet another embodiment, the present invention is directed to a food composition comprising one or more ingredients suitable for consumption by human and the composition described in this application.

In another embodiment, the present invention is directed to the use of the above described composition in treating urinary tract infection and vaginal infection. In one embodiment, the composition which comprises at least 5 billion CFUs of the probiotic mixture and at least 4.7×10⁵ PFU of the phage mixture is administered orally to the women once a day. In one embodiment, the treatment regimen lasts at least 1 week, 2 weeks, 3 weeks, 4 weeks, 1 month, 2 months, 3 months, 4 months, 5 months, or 6 months. In one embodiment, the pathogenic E. coli population in the urinary tract of individuals who take the above mentioned composition is reduced by at least 1, at least 2, at least 3, at least 4, at least 5, or at least 6 log unit (multiple of 10) lower than that of the individuals who take the same amount of phage mixture alone.

In another embodiment, the present invention is directed to the use of the above described composition in improving the vaginal and/or fecal microbiota of a woman. In one embodiment, the composition which comprises at least 5 billion CFUs of the probiotic mixture and at least 5×10⁵ PFU of the phage mixture is administered orally to the women once a day. In one embodiment, the treatment regimen lasts at least 1 week, 2 weeks, 3 weeks, 4 weeks, 1 month, 2 months, 3 months, 4 months, 5 months, or 6 months.

In another embodiment, the present invention is directed to the use of the above described composition in increasing the population of vaginal lactobacilli of a woman. In one embodiment, the composition which comprises at least 5 billion CFUs of the probiotic mixture and at least 5×10⁵ PFU of the phage mixture is administered orally to the women once a day. In one embodiment, the treatment regimen lasts at least 1 week, 2 weeks, 3 weeks, 4 weeks, 1 month, 2 months, 3 months, 4 months, 5 months, or 6 months. In one embodiment, the vaginal lactobacilli population in the women who take the above mentioned composition is increased by at least 1, or at least 2 fold (multiple of 2) higher than that of the women who take the same amount of probiotics mixture alone.

In another embodiment, the present invention is directed to the use of the above described composition in improving or maintaining the urinary tract health and vaginal health of a woman. In one embodiment, the composition which comprises at least 5 billion CFUs of the probiotic mixture and at least 5×10⁵ PFU of the phage mixture is administered orally to the women once a day. In one embodiment, the treatment regimen lasts at least 1 week, 2 weeks, 3 weeks, 4 weeks, 1 month, 2 months, 3 months, 4 months, 5 months, or 6 months.

In another embodiment, the present invention is directed to the use of the above described composition in improving the vaginal and/or fecal microbiota of a woman. In one embodiment, the composition which comprises at least 5 billion CFUs of the probiotic mixture and at least 5×10⁵ PFU of the phage mixture is administered orally to the women once a day. In one embodiment, the treatment regimen lasts at least 1 week, 2 weeks, 3 weeks, 4 weeks, 1 month, 2 months, 3 months, 4 months, 5 months, or 6 months.

In this specification and in the claims that follow, reference will be made to a number of terms, which shall be defined to have the following meanings:

As used herein the term “female microbiota” refers the combined female vaginal microbiota, gastrointestinal microbiota, urinary tract microbiota, and perineum microbiota.

As used herein the term “selectivity” refers to a difference in inhibitory activity towards commensal bacteria and against pathogenic bacteria and/or opportunistic pathogenic bacteria.

Conveniently the level of selectivity may be represented numerically by comparing suitable quantified levels of inhibition, such as minimum inhibitory concentrations (MIC), such as MIC, MIC₅₀ or MIC₉₀ values. The comparison is typically made between two different species, but may also be made between strains within the same species. The comparison may be represented as a ratio of:(the inhibitory activity towards a commensal bacterial species) to (the inhibitory ratio against a pathogenic bacterial species); or (the inhibitory activity towards a commensal bacterial species) to (the inhibitory ratio against an opportunistic pathogenic bacterial species).

As used herein the terms “inhibit”, “inhibition”, “inhibitory”, particularly with respect to microorganism growth, refer to a decrease in the rate of growth of the microbial species with reference to the uninhibited rate of growth of the microbial species. Typically bacterial growth can be measured by counting the change in the number of cells as a function of time, although other methods such as medium digestion, metabolite production, etc. are envisaged. In some embodiments, the degree of inhibition is determined by measuring the difference in rate of growth of a population of a bacterial species as a function of time as compared to a different population of the bacterial species grown in the same conditions without the inhibitor, such as the combination of the present invention.

The terms “treatment” and “treating” as used herein cover any treatment of an infection in an animal, preferably a human, and includes: (i) inhibiting the infection; (ii) relieving the infection; or (iii) relieving the conditions caused by the infection, eg symptoms of the infection.

The terms “prevention” and preventing” as used herein cover the prevention or prophylaxis of an infection in an animal, preferably a human and includes preventing the infection from occurring in a subject which may be predisposed to the infection but has not yet been diagnosed with the infection.

As used herein, the term “synergistic” means that the effect achieved with the compositions of the invention is greater than the sum of the effects that result from using the individual components as a monotherapy. Advantageously, such synergy provides greater efficacy at the same doses.

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein.

“Optional” or “optionally” means that the subsequently described event or circumstance can or cannot occur, and that the description includes instances where the event or circumstance occurs and instances where it does not.

A “probiotic”, as used herein, is an oral supplement or a food product that contains a sufficient number of viable microorganisms to deliver beneficial health effects.

A “probiotic powder”, as used herein, is the actual bacteria and/or yeast in dry form along with any nutrients included for the purpose of sustaining the colonies once they are activated.

“Probiotic concentration” is the amount of colony forming units or CFUs per gram of the probiotic product.

“Phage concentration” is the amount of phage forming units or PFUs per gram of the phage product.

A “administered dose amount” is the volume of powder-containing suspension administered to the women.

Unless defined otherwise, all technical and scientific terms used herein have the same meaning as commonly understood by one of ordinary skill in the art to which this invention belongs.

Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well-known and commonly employed in the art.

The features and advantages of the invention may be more readily understood by those of ordinary skill in the art upon reading the following detailed description. It is to be appreciated that certain features of the invention that are, for clarity reasons, described above and below in the context of separate embodiments, may also be combined so as to sub-combinations thereof.

Embodiments identified herein as exemplary are intended to be illustrative and not limiting.

### EXAMPLES

### Example 1

In this study, in vitro culture studies were conducted to determine the ability of phage blend to inhibit or reduce the growth of bacteria and thus enhances the growth of probiotics that benefit host well-being and health.

All studies were done under physiological conditions of human body (37° C. & pH 6.8). The bacteria selected for testing included E. coli and three probiotic strains of Bifidobacterium breve, Lactobacillus plantarum, and Bifidobacterium lactis. The phage blend contains Myoviridae strains LH01, T4D, and LL12, and Siphoviridae strain LL5, and was used at 15 mg.

Bacterial cells from each strain were incubated in 50 mls of nutrient broth (1% glucose) until the culture reached an optical density of 0.2. For competition experiments, each bacterium was grown to an O.D. of 0.2, halved and added together. The phage blend was quickly added at 15 mg to one set and broth to the other which served as the control. For anaerobic growth, 0.2 cultures were divided into flasks and purged with a 5 second shot of nitrogen, both the phage blend and broth were divided up and added to each flask, sealed and put back on the shaker. The anaerobic growth condition used here denotes low instead of completely without oxygen, which reflects the atmosphere of the human body. Anaerobic samples were opened once, tested and discarded. Samples containing probiotic alone, E. coli alone, E. coli and phage blend and phage blend alone, were mixed and allowed to grow for the indicated time period in a shaker bath at 37° C. and pH 6.8. One milliliter sample of each mixture was taken and diluted several times in Hardy Diagnostic phosphate buffer and plated on selected media for each type of probiotic. The E. coli sample was plated on 3M E. coli plates and the selected media alone as a control. E. coli that grew on the selected media for the probiotic was subtracted from the total plate count of the mixture. MRS agar (made by Neogen) plates were used for quantification of Lactobacillus strains, Bifidobacterium agar (made by Hardy Diagnostics) was used for the quantification of Bifidobacterium strains. Experiments were all done in triplicate, multiple dilutions were also performed and 5 or more plates were averaged to determine bacterial counts.

Lactobacillus bacterial counts per milliliter after growth for 10 hours with and without the phage blend. Bifidobacterium bacterial counts per milliliter are represented below after growth for 10 hours with and without the phage blend.

TABLE 1

| | Bacterial counts |
|---|---|
| Composition | (CFU per milliliter) |
| Lactobacillus plantarum alone | 4100 |
| Lactobacillus plantarum + the phage blend | 16000 |
| Bifidobacterium breve alone | 2600 |
| Bifidobacterium breve + the phage blend | 35000 |
| Bifidobacterium lactis alone | 1900 |
| Bifidobacterium lactis + the phage blend | 6200 |

The bacteria counts shown in Table 1 is the cell count of probiotics such as Bifidobacterium breve, Lactobacillus plantarum, and Bifidobacterium lactis, because the bacteria counts were calculated by subtracting the cell count of E. coli that grew on the selected media from the total plate count of the mixture of E. coli and the probiotics. It was observed from the data in Table 1 that the mixture of probiotic and the phage blend have a much higher number of surviving probiotic cell counts when they are co-incubated with E. coli, presumably because the phage blend killed the E. coli and/or inhibited their growth. The presence of the phage blend with beneficial probiotic strains of Lactobacillus and Bifidobacterium enhanced the growth of these bacteria probiotic strains.

### Example 2

In this study, the effect of the phage blend for killing E. coli and enhancement of the growth of beneficial bacteria was tested in another set of probiotic strains.

The study conducted under physiological conditions of human body (37° C. & pH 6.8). The bacteria selected for testing included E. coli and a blend of 4 probiotic strains of Lactobacillus crispatus strain LBV 88, Lactobacillus rhamnosus strain LBV 96, Lactobacillus gasseri strain LBV 150N, Lactobacillus jensenii strain LBV 116. The phage blend contains Myoviridae strains LHO1, T4D, and LL12, and Siphoviridae strain LL5, and was used at 15 mg.

Bacterial cells of several fresh colonies were incubated in 50 mL of nutrient broth (1% glucose) until the culture reached an optical density (OD) of 0.2. For competition experiments, each bacterium was grown to an OD of 0.2, halved and added together. The phage blend was quickly added at 15 mg to one set and broth to the other which serves as the control. For anaerobic growth, 0.2 OD cultures were divided into approximately 36 flasks; each purged with a 5 second shot of nitrogen. Half of the flasks were inoculated with the phage blend and the remaining were inoculated with broth (control) each was then sealed and put back on the shaker. The anaerobic growth condition in this experiment denotes low (<10%) instead of completely without oxygen, which reflects the atmosphere of the human body.

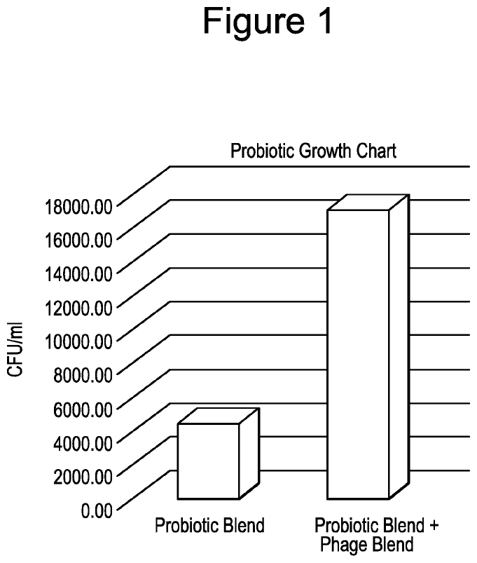

Anaerobic samples are tested as follows:
Each sample is opened once, tested and discarded.
Three probiotic blend samples are tested alone.
Three E. coli samples are tested alone.
Fifteen probiotic blend+E. coli samples are tested (shown in FIG. 1).
Fifteen probiotic blend+E. coli+ the phage blend together (shown in FIG. 1).

Each are mixed and allowed to grow for indicated time period (5 hours) in a shaker bath at 37° C. and pH 6.8.

It was observed from the data in FIG. 1 that the mixture of probiotic blend and the phage blend have a much higher number of surviving probiotic cell counts than the probiotic blend alone when they were co-incubated with E. coli. This data confirms the observation made in Example 1. It suggests that phage blend not only killed the E. coli pathogen, but also enhanced the growth of beneficial probiotic bacterial strains.

### Example 3

In this study, the effect of the phage blend to kill E. coli and enhances the growth of beneficial bacteria was tested in a mice model.

The clinically isolated enterotoxigenic E. coli (ETEC) strain, H10407 (serotype 078:H11) was used to represent the major serotypes isolated worldwide from major microbial imbalances.

This enterotoxigenic E. coli strain H10407 was originally isolated in Bangladesh from a patient with severe, cholera-like diarrheal illness. It was derived from good manufacturing practice (GMP) lots of H10407 produced at Walter Reed Army Institute of Research. This strain is fully virulent in human volunteer clinical challenge studies.

A mice model has infection of pathogenic E. coli strain was created. Mice were infected orally with enterotoxigenic E. coli strain H10407 as previously described by Allen et al. 2006, Infect. Immun. 74:869-875. Briefly, strain H10407 was grown to mid-logarithmic phase in Luria broth, pH 7.4, and resuspended in sterile PBS such that the final concentration of bacteria was approximately 5×10⁷ CFU per dose plus 2.5×10⁷ CFU per dose of Bifidobacterium longum in a final volume of 300:1. This amount was then administered by gavage to 12 ETEC-naïve ICR mice that had been pretreated with streptomycin to eliminate native flora and cimetidine to reduce stomach acidity prior to challenge. This procedure was repeated with the addition of 1×10⁵ plaque forming units (PFU) per dose of the E. coli phage blend. Fecal matter was taken 2 times at 6 and 24 hours after inoculation and mice were subsequently sacrificed at 24 hours. The ileum and large intestine were harvested and plated for E. coli counts, B. longum counts and phage counts.

The result shows that the addition of phage blend into a mixture of pathogenic E. coli and beneficial bacteria probiotic strain B. longum resulted a significantly decreased titer pathogenic E. coli and a significantly increased titer of good bacteria probiotic strain B. longum.

It was observed that the E. coli decreased in the ileum ˜10 fold (changing from 50170 PUF without the phage blend to 3135 PFU with the phage blend), in the large intestine ˜100 fold (changing from 11180 PUF without the phage blend to 49 PFU with the phage blend) and in the fecal matter ˜150 fold (changing from 10525 PUF without the phage blend to 67 with the phage blend) at 24 hours.

The B. longum counts increased ˜100 fold in the ileum (changing from 40423 PFU without the phage blend to 73 PFU with the phage blend), ˜100 fold in the large intestine (changing from 1001 PFU without the phage blend to 12 PFU with the phage blend), and ˜34 fold in the 24 hour fecal sample (changing from 18050 PFU without the phage blend to 505 PFU with the phage blend).

Phage counts went up in the ileum from 897 PFU with B. longum only to 51150 PFU with E. coli and B. longum. Then in the large intestines phage counts went up from 695 PFU to 91500 PFU. In the 24 hour fecal count, the phage counts increased from 582 PFU to 87000 PFU.

Mice that were inoculated with E. coli alone and E. coli and B. longum combo showed constipation and their ileum, cecal valve and large intestine were swollen, red and leaking when compared to the control mice with no inoculation. The mice that were inoculated with E. coli and the phage blend exhibited normal bowel movements and experienced no change in color or size of the various compartments of the intestine when compared with control mice.

It was therefore shown above that an oral phage blend decreased intestinal pathogenic E. coli populations from 10-1000 fold while simultaneously increasing probiotic populations by 10-100 fold. The increase in probiotic counts reflects the decrease in competition and release of nutrients from the pathogenic E. coli bacteria. These in vivo results strongly suggested that addition of a phage and probiotic blend helps to regulate and enhance the intestinal microflora.

### Example 4

This example describes the evaluation of the effect of the probiotic blend (PB), bacteriophage blend (BB), and the combination of both on the development of Escherichia co/i (EC). The EC strain used in this example was isolated from urine from a patient with a urinary tract infection. Multilocus sequence typing using the Achtman scheme identified this isolate as sequence type ST2491 which is assigned to the ST10 clonal complex. This strain was deposited in the culture collection of the Westerdijk Fungal Biodiversity Institute and is available as CBS 147900.

The effect of PB, BB, and the combination of both on the development of EC was evaluated in Plate Count Broth with additional CaCl₂. This medium which is referred to below as PCB had the following composition: 5 g/l yeast extract, 10 g/l tryptone, 2 g/l glucose, 1 mM CaCl₂. Tests were done at pH 7.0 and pH 5.0.

250 mg PB (CSL starter mix, product nr G001411, batch E014797A) was dissolved in 50 ml sterile PCB and resuscitated for 1 hour at room temperature. After resuscitation, PB was diluted 100-fold in PCB to a final volume of 50 ml.

500 mg BB (PreforPro®, product nr 25203B, 5×10⁸ PFU/g, Deerland Probiotics & Enzymes) was dissolved in 50 ml PCB. BB suspension was subsequently filter-sterilized (Acrodisc Supor® filter, low protein binding, pore size 0.45 μm, Pall).

EC strain CBS 147900 was cultivated in PCB pH 7.0 without additional CaCl₂ for 23±1 h at 35° C. After cultivation the culture was stepwise diluted 2,500-fold in PCB to a final volume of 50 ml.

The effect of PB, BB, and the combination of both on the development of EC was evaluated in 24 well microtiter plates (sterile, VWR). The following mixtures were prepared in triplicate in the microtiter plates:

### (1) 500 μl EC, 1.5 ml PCB

### (2) 500 μl PB, 1.5 ml PCB

### (3) 500 μl BB, 1.5 ml PCB

### (4) 500 μl EC, 500 μl PB, 1 ml PCB

### (5) 500 μl EC, 500 μl BB, 1 ml PCB

### (6) 500 μl PB, 500 μl BB, 1 ml PCB

### (7) 500 μl EC, 500 μl PB, 500 μl BB, 500 μl PCB

The microtiter plates were covered with sterile sealing tape (MicroWell™, Nunc™ Thermo Scientific) to prevent evaporation and incubated at 35° C.

Immediately after the preparation of the mixtures (0 h) and during the incubation (after 24, 48 and 72 or 96 h), samples were taken from the mixtures to determine the viable count (colony forming units per ml; CFU/ml) of EC by plate counting. Tryptone Soya Agar (TSA) was used for the enumeration of EC. TSA plates were incubated aerobically at 35° C. for 1 day. Preparatory tests had shown that the selected cultivation condition for EC did not yield visible colonies of PB. The limit of quantification was 100 CFU/ml. Viable counts are expressed as their log₁₀ value (log CFU/ml). Statistical analysis was done with the Minitab 18 software tool using One Way ANOVA (95% confidence level) and Tukey's method for pairwise comparisons as post hoc test.

The viable counts of EC during the incubations in PCB pH 7.0 are shown in Table 2. Unless stated otherwise, the values in these tables represent the mean viable count ±standard deviation of 3 replicate mixtures. The letters following the standard deviation refer to the groups that were identified using Tukey's method for pairwise comparisons. Means within a column that do not share the same letter, are significantly different (p<0.05).

EC strain CBS 147900 grew well in PCB, resulting in cell densities between 9 and 10 log CFU/ml. At pH 7.0, PB alone did not have any effect on the viable count of EC throughout the incubation while with BB only a moderately inhibitory effect was observed after 72 h (Table 2). In contrast, the combination of PB and BB had a strongly inhibitory effect on the development of EC. This effect became apparent after 48 h incubation, and after 72 h the combination of PB and BB had resulted in a viable count of EC which was more than 6 log units lower than in the incubation with EC alone, and more than 4 log units lower than in the incubation with EC and BB.

TABLE 2

| | | Viable count EC | | | |
|---|---|---|---|---|---|
| | | (log CFU/ml) | | | |
| Mixture | Additions | 0 h | 24 h | 48 h | 72 h |
| 1 | EC | 4.8 ± 0.05 A | 9.5 ± 0.06 A | 9.0 ± 0.02 A | 9.1 ± 0.20 A |
| 4 | EC, PB | 4.9 ± 0.11 A | 8.7 ± 0.16 A | 8.5 ± 0.07 A | 8.4 ± 0.09 AB |
| 5 | EC, BB | 4.9 ± 0.10 A | 6.2 ± 0.26 B | 8.3 ± 0.34 A | 7.7 ± 0.54 B |
| 7 | EC, PB, BB | 4.8 ± 0.06 A | 5.3 ± 0.89 B | 4.8 ± 1.2 B | ≤3.0* C |

*This value represents the highest viable count of the 3 replicate mixtures. In the other 2 mixtures, the viable counts were below the limit of quantification.

Table 3 shows the reduction of the viable count of EC strain CBS 147900 in PCB pH 7.0 by PB and BB separately and combined. In this table, the reduction of the viable count of EC in PCB pH 7.0 that is achieved by the combination of PB and BB is compared to the sum of the effect of PB and BB separately. From this table it is obvious that the effect of the combination of PB and BB that is observed at 48 h and 72 h is achieved by synergistic interaction of PB and BB since the reduction of the viable count is larger than the sum of the reductions obtained with PB and BB separately.

TABLE 3

| | Reduction of viable count EC | | |
|---|---|---|---|
| | (log units) after | | |
| Addition | 24 h | 48 h | 72 h |
| PB | 0.8 | 0.5 | 0.7 |
| BB | 3.2 | 0.7 | 1.4 |
| PB and BB | 4.1 | 4.2 | ≥6.1 |
| Sum of the effect | 4.0 | 1.2 | 2.1 |
| of PB and BB | | | |
| separately | | | |

The viable counts of EC during the incubations in PCB pH 5.0 are shown in Table 4, respectively. The values in these tables represent the mean viable count ±standard deviation of 3 replicate mixtures. The letters following the standard deviation refer to the groups that were identified using Tukey's method for pairwise comparisons. Means within a column that do not share the same letter, are significantly different (p<0.05).

At pH 5, presence of PB or BB alone resulted in a moderate inhibition of the viable count of EC strain CBS 147900 after 96 h incubation (Table 4). With BB this effect became visible after 48 h incubation while with PB this effect became visible only at the end of the incubation. In contrast, the combination of PB and BB had a strongly inhibitory effect on the development of EC. After 48 h incubation, this inhibitory effect was still moderate and not significantly different from the effect of BB alone. However, after 96 h incubation, no viable cells of EC were detected anymore in each of the mixtures that contained the combination of PB and BB, corresponding to more than 7.4 log units reduction compared to the control mixtures that contained only EC.

TABLE 4

| | | Viable count EC | | | |
|---|---|---|---|---|---|
| | | (log CFU/ml) | | | |
| Mixture | Additions | 0h | 24 h | 48 h | 96 h |
| 1 | EC | 4.7 ± 0.09 A | 8.9 ± 0.04 A | 9.7 ± 0.74 A | 9.4 ± 0.37 A |
| 4 | EC, PB | 4.7 ± 0.10 A | 8.7 ± 0.14 A | 9.1 ± 0.32 A | 7.7 ± 1.3 B |
| 5 | EC, BB | 4.7 ± 0.06 A | 8.2 ± 0.08 A | 7.8 ± 0.13 B | 7.4 ± 0.29 B |
| 7 | EC, PB, BB | 4.7 ± 0.07 A | 8.3 ± 0.07 A | 7.3 ± 0.10 B | <2 C |

In Table 5, the reduction of the viable count of EC strain CBS 147900 in PCB pH 5.0 that is achieved by the combination of PB and BB, is compared to the sum of the effect of PB and BB separately. From this table it is clear that the effect of the combination of PB and BB that is observed at 96 h is achieved by synergistic interaction of PB and BB since the reduction of the viable count is larger than the sum of the reductions obtained with PB and BB separately.

TABLE 5

| | Reduction of viable count EC | | |
|---|---|---|---|
| | (log units) after | | |
| Addition | 24 h | 48 h | 96 h |
| PB | 0.3 | 0.5 | 1.7 |
| BB | 0.7 | 1.8 | 1.9 |
| PB and BB | 0.6 | 2.4 | >7.4 |
| Sum of the effect | 0.9 | 2.4 | 3.6 |
| of PB and BB | | | |
| separately | | | |

### Example 5

This example describes the evaluation of the effect of the probiotic blend (PB), bacteriophage blend (BB), and the combination of both on the development of Escherichia coli (EC). The EC strain used in this example was isolated from urine from a patient with a urinary tract infection. Multilocus sequence typing using the Achtman scheme identified this isolate as sequence type ST1453 which is assigned to the ST73 clonal complex. This strain was deposited in the culture collection of the Westerdijk Fungal Biodiversity Institute and is available as CBS 147899.

The effect of PB, BB, and the combination of both on the development of EC was evaluated in Plate Count Broth with additional CaCl₂. This medium is referred to below as PCB. Tests were done at pH 7.0 and pH 5.0. The experimental approach and details are described in Example 4.

The viable counts of EC during the incubations in PCB pH 7.0 are shown in Table 6, respectively. The values in these tables represent the mean viable count ±standard deviation of 3 replicate mixtures. The letters following the standard deviation refer to the groups that were identified using Tukey's method for pairwise comparisons. Means within a column that do not share the same letter, are significantly different (p<0.05).

EC strain CBS 147899 grew well in PCB, resulting in cell densities between 8 and 9 log CFU/ml (Table 6). Throughout the incubation, PB and BB separately did not have any inhibitory effect on the viable count of EC. In contrast, the combination of PB and BB had a very strongly inhibitory effect on the development of EC. After 48 h incubation, the combination of PB and BB had resulted in a viable count of EC which was more than 6.5 log units lower than in the incubation with EC alone, and after 72 h incubation, no viable cells of EC were detected anymore in each of the triplicate incubations.

TABLE 6

| | | Viable count EC | | | |
|---|---|---|---|---|---|
| | | (log CFU/ml) | | | |
| Mixture | Additions | 0 h | 24 h | 48 h | 72 h |
| 1 | EC | 4.7 ± 0.03 A | 8.3 ± 0.10 A | 8.5 ± 0.20 A | 8.5 ± 0.10 A |
| 4 | EC, PB | 4.6 ± 0.10 A | 8.3 ± 0.08 AB | 8.2 ± 0.07 B | 8.1 ± 0.11 B |
| 5 | EC, BB | 4.6 ± 0.04 A | 8.0 ± 0.07 BC | 8.1 ± 0.07 B | 8.4 ± 0.13 A |
| 7 | EC, PB, BB | 4.7 ± 0.05 A | 7.9 ± 0.08 C | ≤2.9* C | <2 C |

*This value represents the highest viable count of the 3 replicate mixtures. In the other 2 mixtures, the viable counts were below the limit of quantification.

In Table 7, the reduction of the viable count of EC strain CBS 147899 in PCB pH 7.0 that is achieved by the combination of PB and BB is compared to the sum of the effect of PB and BB separately. From this table it is obvious that the effect of the combination of PB and BB that is observed at 48 h and 72 h is achieved by synergistic interaction of PB and BB since the reduction of the viable count is larger than the sum of the reductions obtained with PB and BB separately.

TABLE 7

| | Reduction of viable count EC | | |
|---|---|---|---|
| | (log units) after | | |
| Addition | 24 h | 48 h | 72 h |
| PB | 0.1 | 0.3 | 0.4 |
| BB | 0.3 | 0.4 | 0.1 |
| PB and BB | 0.4 | >5.6 | >6.5 |
| Sum of the effect | 0.4 | 0.7 | 0.5 |
| of PB and BB | | | |
| separately | | | |

The viable counts of EC during the incubations in PCB pH 5.0 are shown in Table 8, respectively. Unless stated otherwise, the values in these tables represent the mean viable count ±standard deviation of 3 replicate mixtures. The letters following the standard deviation refer to the groups that were identified using Tukey's method for pairwise comparisons. Means within a column that do not share the same letter, are significantly different (p<0.05).

At pH 5.0, BB alone did not have any inhibitory effect on the viable count of EC throughout the incubation (Table 8). In contrast, PB alone and the combination of PB and BB had a strongly inhibitory effect on the development of EC. After 72 h incubation, no viable EC cells were detected anymore in all 3 mixtures that contained both PB and BB and in 2 of the 3 mixtures that were inoculated with PB alone. Since the presence of PB alone already had such a large effect on the viability of EC, this experiment could not answer the question whether the effect observed with the combination of PB and BB was the result of synergistic interaction or should be attributed to PB alone.

TABLE 8

| | | Viable count EC | | | |
|---|---|---|---|---|---|
| | | (log CFU/ml) | | | |
| Mixture | Additions | 0 h | 24 h | 48 h | 72 h |
| 1 | EC | 4.5 ± 0.09 A | 7.8 ± 0.19 A | 8.3 ± 0.14 A | 7.1 ± 0.17 A |
| 4 | EC, PB | 4.5 ± 0.06 A | 6.8 ± 0.36 B | —* | ≤3.6 B** |
| 5 | EC, BB | 4.6 ± 0.06 A | 8.0 ± 0.04 A | 8.0 ± 0.20 A | 7.3 ± 0.07 A |
| 7 | EC, PB, BB | 4.6 ± 0.15 A | 6.8 ± 0.11 B | ≤3.9 B** | <2 B |

*No data available due to unintended deviation from protocol.
**This value represents the highest viable count of the 3 replicate mixtures. In the other 2 mixtures, the viable counts were below the limit of quantification.

### Example 6

In this double-blind, randomized, controlled trial, the effect of four probiotic Lactobacillus strains (L. crispatus, L. gasseri, L. rhamnosus, L. jensenii) and a bacteriophage blend are evaluated against E. coli strains on gut and vaginal microbiota, prebiotic properties, safety and recurrence of urinary tract infection (UTI) in adult women.

Women aged≥18 y with stable menstrual cycle or postmenopausal women having experienced a symptomatic UTI within the last 4 weeks were included in the study. Women are given once daily for 6 months a capsule containing living strains of L. crispatus LbV 88 (DSM 22566), L. gasseri LbV 150N (DSM 22583), L. jensenii LbV 116 (DSM 22567) and L. rhamnosus LbV96 (DSM 22560)), in a concentration of 5×10⁹ CFU/g with a blend of bacteriophages or a placebo capsule.

The primary target parameter was recurrence of symptomatic UTI. Secondary target parameters included severity of UTIs as assessed by UTISA questionnaire and duration of UTIs. Exploratory parameters included incidence of E. coli infections of the UT, alteration of vaginal pH, vaginal pH at G2, alterations in microbiota and adverse events.

At the end of the 6 month period a reduction in urinary tract infections is demonstrated in the probiotic and phage group. This reduction in UTIs is not demonstrated in the placebo group. The severity and duration of urinary tract infections is also improved in the treatment group and not in the placebo group. Improvements in the microbiota are also demonstrated in the treatment group and not in the placebo group.

1. A composition comprising at least one probiotic which has anti-bacterial vaginosis and/or anti-candida activity and at least one phage which has anti-pathogenic E. coli activity.

2. The composition of claim 1, wherein said probiotic comprises a prokaryote, eukaryote, or archaebacteria probiotic.

3. The composition of claim 2, wherein said probiotic comprises at least one of any suitable strain or subspecies of Enterococcus, Streptococcus, Lactobacillus, Lactococcus, Bifidobacterium, or Saccharomyces.

4. The composition of claim 3, wherein said probiotic comprises Lactobacillus crispatus strain LBV 88, Lactobacillus rhamnosus strain LBV 96, Lactobacillus gasseri strain LBV 150N, and Lactobacillus jensenii strain LBV 116.

5. The composition of claim 1, wherein said phage comprises at least one of suitable strain in the families of Myoviridae and Siphoviridae.

6. The composition of claim 5, wherein said phage comprises Myoviridae strains LHO1, T4D, and LL12, and Siphoviridae strain LL5.

7. The composition of claim 1, wherein the composition is formulated in a dosage which is sufficient for improving or maintaining the urinary tract health and vaginal health of a woman.

8. The composition of claim 1, wherein the composition is formulated in a dosage which is sufficient for treating, reducing the duration and severity, or reducing the risk of urinary tract infection and vaginal infection of a woman.

9. The composition of claim 1, wherein the composition is formulated in a dosage which is sufficient for improving the vaginal and/or fecal microbiota of a woman.

10. The composition of claim 1, wherein the composition is formulated in a dosage which is sufficient for increasing the population of vaginal lactobacilli of a woman.

11. The composition of claim 1, wherein the probiotic in said composition is formulated in a dosage of more than 5 billion CFUs per day.

12. The composition of claim 11, wherein the phage in said composition is formulated in a dosage of more than 4.7×10⁵ PFU per day.

13. The composition of claim 12, wherein said composition is formulated for oral administration.

14. The composition of claim 13, wherein said composition is formulated as a dietary supplement, a food, a medical food, or a pharmaceutical.

15. A kit suitable for administering a composition orally to a human, comprising in a packet the composition of claim 1, and wherein said composition is in the form of powder, and instructions for how to use the kit.

16. A method for treating woman's urinary tract infection and/or vaginal infection of a woman, comprising orally administering to the woman the composition of claim 1.

17. A method for reducing the duration of severity of woman's urinary tract infection and/or vaginal infection of a woman, comprising orally administering to the woman the composition of claim 1.

18. A method for improving the vaginal and/or fecal microbiota of a woman, comprising orally administering to the woman the composition of claim 1.

19. A method for increasing the population of vaginal lactobacilli of a woman, comprising orally administering to the woman the composition of claim 1.

20. A method for improving or maintaining the urinary tract health and vaginal health of a woman, comprising orally administering to the woman the composition of claim 1.
